(19) United States
(12) Patent Application Publication
(10) Pub. No.: US 2024/0398224 A1
(43) Pub. Date: Dec. 5, 2024

(54) EYE TRACKING USING COHERENCE-BASED MEASUREMENT

(71) Applicant: APPLE INC., Cupertino, CA (US)

(72) Inventors: Walter NISTICO, Redwood City, CA (US); Avery L. WANG, Palo Alto, CA (US); Edward VAIL, Menlo Park, CA (US); Huiyang DENG, San Jose, CA (US); Samuel STEVEN, Cupertino, CA (US); Seyedeh Mahsa KAMALI, San Jose, CA (US); Tong CHEN, Fremont, CA (US); Xibin ZHOU, Palo Alto, CA (US)

(21) Appl. No.: 18/693,276

(22) PCT Filed: Sep. 20, 2022
(86) PCT No.: PCT/US2022/044060

§ 371 Date: Mar. 19, 2024

Related U.S. Application Data
(60) Provisional application No. 63/248,062, filed on Sep. 24, 2021.

(51) Int. Cl. A61B 3/00; A61B 3/10; A61B 3/11

(52) U.S. Cl. CPC A61B 3/0025; A61B 3/102; A61B 3/112

(57) ABSTRACT

Various implementations disclosed herein include devices, systems, and methods that track a state of a user's eye (e.g., position/orientation, gaze direction accommodation, pupil dilation, etc.) using coherence-based measurement (e.g., optical coherence tomography (OCT)). The coherence-based measurement may provide sub-surface information, e.g., depth, cross section, or a volumetric model of the eye, based on reflections/scattering of light (e.g., using relatively long wavelength light to penetrate into the eye tissue).

### TECHNICAL FIELD

The present disclosure generally relates to tracking eye characteristics such as eye position/orientation, gaze direction, accommodative state, and pupil dilation, and in particular, to systems, methods, and devices for tracking eye characteristics based on coherence-based measurements.

### BACKGROUND

Some existing systems use light reflected off front surfaces of the eye to estimate eye characteristics. For example, such techniques may estimate the user's gaze direction using multiple glints reflected off the front eye surface to identify locations along the user's gaze (e.g., pupil center, eye center, and cornea center). Other techniques use images of a retina to track eye characteristics. The robustness, accuracy, and/or efficiency of existing eye tracking techniques may be improved.

### SUMMARY

Various implementations disclosed herein include devices, systems, and methods that track a state of a user's eye (e.g., eye position/orientation, gaze direction accommodation, pupil dilation, etc.) using coherence-based measurement (e.g., optical coherence tomography (OCT)). The coherence-based measurement may provide sub-surface information, e.g., depth, cross section, or a volumetric model of the eye, based on reflections/scattering of light (e.g., using relatively long wavelength light to penetrate into the eye tissue).

Some implementations involve a method of determining eye characteristics at an electronic device having a processor. The processor may execute instructions stored in a non-transitory computer-readable medium to implement the method. The method may obtain a three-dimensional (3D) representation of an eye (e.g., a 3D point cloud or other 3D model of the user's eye based on a prior scan or a standard eye model based on population averages). The method generates a scan including information about the thickness or 3D volumetric structure of a portion of an eye. The scan involves detecting interference between a split off reference wave and a reflection or scattering by sample of an incident wave. In some implementations, the scan occurs in multiple stages, for example, to conserve device resources. Such a multi-stage scan may involve an initial fine scan (e.g., dense B-scan) followed by coarse scan (e.g., sparse A-scan). In some implementations, the scan identifies a sparse set of points corresponding to thickness or 3D volumetric structure of the eye portions (e.g., intercepts with cornea front/back surfaces, iris surfaces, pupil contour points, crystallin lens surfaces, ciliary muscles, points on retina or choroid, or retinal blood vessels). The method tracks the eye based on the scan by comparing the scan with the 3D representation of the eye. As examples, the method may track the 3D position and orientation of the eye (e.g., eye pose), gaze direction, accommodative state, pupil dilation state, physiological state, health conditions, etc.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an example operating environment in accordance with some implementations.

FIG. 2 illustrates an example head-mounted device (HMD) in accordance with some implementations.

FIG. 3 illustrates an exemplary coherence-based measurement on an exemplary eye in accordance with some implementations.

FIG. 4 illustrates data from an exemplary coherence-based measurement of an eye in accordance with some implementations.

FIG. 5 illustrates data from an exemplary coherence-based measurement of an eye in accordance with some implementations.

FIG. 6 illustrates ciliary changes corresponding to different accommodative states that may be detected in accordance with some implementations.

FIG. 7 is a flowchart representation of a method of tracking eye characteristics in accordance with some implementations.

FIG. 8 illustrates components of an exemplary device in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects and/or variants do not include all of the specific details described herein. Moreover, well-known systems, methods, components, devices and circuits have not been described in exhaustive detail so as not to obscure more pertinent aspects of the example implementations described herein.

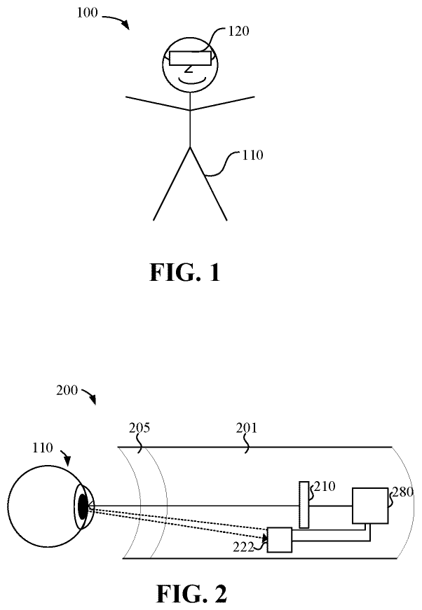

FIG. 1 is a block diagram of an example operating environment 100. In some implementations, the user wears the device 120 on his/her head. As such, the device 120 may include one or more displays configured to display content. The device 120 may enclose the field-of-view of the user. In some implementations, the device 120 is a handheld electronic device (e.g., a smartphone or a tablet) configured to present content to the user. In some implementations, the device 120 is replaced with a chamber, enclosure, or room configured to present content in which the user does not wear or hold the device 120.

FIG. 2 illustrates an example head-mounted device (HMD) 200 which may be an example of device 120. The HMD 200 includes a housing 201 (or enclosure) that houses various components. The housing 201 includes (or is coupled to) an eye pad 205 disposed at a proximal (to the user 110) end of the housing 201. In various implementations, the eye pad 205 is a plastic or rubber piece that comfortably and snugly keeps the head-mounted device 200 in the proper position on the face of the user 110 (e.g., surrounding the eye of the user 110).

The housing 201 houses a display 210 that displays an image, emitting light towards the eye of a user 110. In various implementations, the display 210 emits the light through an eyepiece (not shown) that refracts the light emitted by the display 410, making the display appear to the user 110 to be at a virtual distance farther than the actual distance from the eye to the display 210. For the user to be able to focus on the display 210, in various implementations, the virtual distance is at least greater than a minimum focal distance of the eye (e.g., 7 cm). Further, in order to provide a better user experience, in various implementations, the virtual distance is greater than 1 meter.

Although FIG. 2 illustrates an HMD 200 including a display 210 and an eye pad 205, in various implementations, the HMD 200 does not include a display 210. In some implementations, the HMD 200 includes an optical see-through display, for example, including a lens or transparent substrate includes a waveguide configured to display virtual content within a view of a physical environment viewed through the lens or transparent substrate.

The housing 201 also houses a tracking system including one or more coherence-based measurement device 222 and a controller 280. The coherence-based measurement device 222 may include a wave source that direct waves (e.g., light) toward the eye of the user 110 at least some of which penetrate the eye's front surfaces and into interior portions of the eye structures and reflects or is scattered by interior aspects of the eye structures. The reflected and/or scattered waves may be detected by the coherence-based measurement device 222 (e.g., a sensor). Based on the reflections/scatterings, the controller 480 can determine an eye characteristic of the user 110, including but not limited to, a gaze direction, accommodation state, pupil dilation state, etc. Thus, in various implementations, the waves are emitted by the one or more wave sources, reflected or are scattered off portions of the eye of the user 110, and are detected by one or more sensors for use in assessing eye characteristics.

In some implementations, the coherence-based measurement device 222 is an optical coherence tomography (OCT) device. The coherence-based measurement device 222 may produce waves (e.g., light) that are directed towards the eye of the user 110, splitting off a portion of the waves for coherence measurement purposes. The coherence-based measurement device 222 may use multiple wavelengths that penetrate the tissue of the eye to varying extents before being reflected or scattered. The reflections or scatterings of the waves are sensed by the coherence-based measurement device 222 by interfering them with the portion of the waves that were split off. A Fourier transform of the wavelength dependent signal that is received to produce data that represents 3D volumetric information, a depth profile, or cross-section of a portion of the eye. Areas of the eye that are similar to water, e.g., interior portions of the eye structures, may reflect or scatter few waves versus areas of the eye, such as the front and back edges of the cornea, which may reflect or scatter relatively more of the waves. In some OCT implementations, the coherence-based measurement device 222 may produce variable or swept frequencies to produce reflections/scatterings at different depths within the tissue. The reflection/scattering of the waves at different depths may thus provide information about the composition of the internal structures of the eye. Accordingly, the coherence-based measurement device 222 may be used to produce data from which a 3D volumetric structure, depth characteristics, and other such attributes can be determined.

The display 210 may emit light in a first wavelength range and the coherence-based measurement device 222 may emit light in a second wavelength range. Similarly, the coherence-based measurement device 222 may detect light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm). In one example, the coherence-based measurement device 222 is configured to produce infrared light centered at a wavelength of about 850 nm.

In various implementations, detected eye characteristics are used to enable user interaction. For example, a detected gaze direction may be used to control a user interface (e.g., the user 110 selects an option on the display 210 by looking at it), provide foveated rendering (e.g., present a higher resolution in an area of the display 210 the user 10 is looking at and a lower resolution elsewhere on the display 210), or reduce geometric distortion (e.g., in 3D rendering of objects on the display 210). Similarly, a detected accommodative state (or user intention to focus at a particular distance), may be used to determine which content a user intends to look at and adjust the user experience accordingly, e.g., by enhancing or changing that content, etc. In another example, a detected pupil dilation state is used to assess a user response to (e.g., interest in) content presented on the display 210.

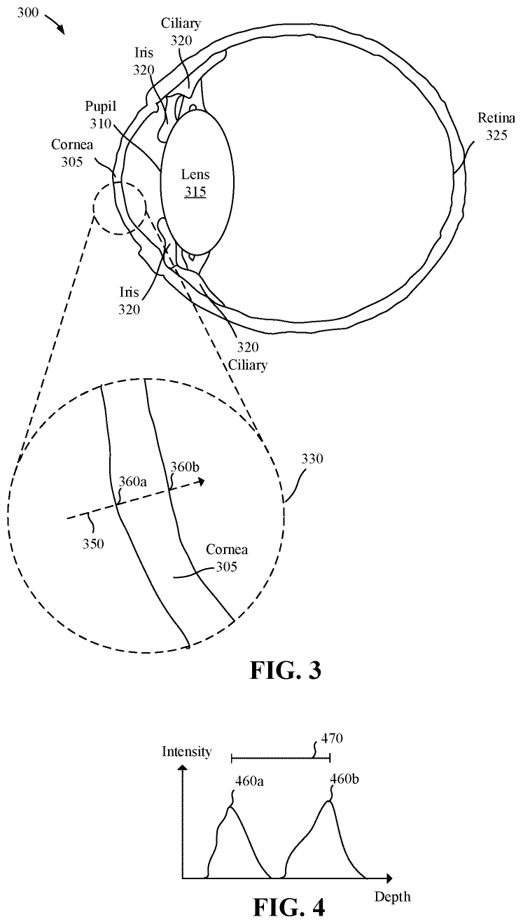

FIG. 3 illustrates an exemplary coherence-based measurement on an exemplary eye 300. In this example, a cross sectional view of the eye 300 is used to illustrate some portions of the eye 300. The eye 300 includes a cornea 305, a pupil 310, a lens 315, an iris 320, ciliary muscles 320, retina 325 and additional portions not shown and/or labelled. In this example, A coherence-measurement is illustrated using a close up view 330 of a portion of the cornea 305. Waves of various wavelengths are directed through the eye 300, e.g., in direction 350. Due the different wavelengths, the waves penetrate the cornea 305 to different degrees before being reflected or scattered by the cornea 305. Some of the reflected or scattered waves are captured by a sensor and used with the original waves (e.g., via a coherence assessment) to determine depth and/or 3D volumetric information of the cornea 305. In one example, such information may be used to determine the depth of the cornea at the position of direction 350 (e.g., at the position of a ray passing into/through the cornea 305 at that location). Rays in multiple directions may penetrate the eye 300 in various directions providing depth information of the eye structures in those various directions.

For example, FIG. 4 illustrates data from an exemplary coherence-based measurement of an eye in along the direction 350. Such an assessment (e.g., an OCT A-scan along a Z/depth axis) may produce an interference signal in which intensity varies for different depths (e.g., there may be more or less reflection/scattering at different depth depending upon the composition and/or structure of the eye portion at those depths). As shown in FIG. 4, the intensity includes a peak 460a and a peak 460b, corresponding to the front surface 360a and back surface 360b of the cornea 305. Such data may be used in various ways. For example, such information may be used to determine the depth 470 of the cornea 305 at the location of the direction 350. Such measurements may be repeated at different location on cornea 305 and for other eye structures to determine depth and/or 3D volumetric information representing many or all of the structures of the eye. Such information may be used to generate a 3D point cloud or other 3D model of the eye 300.

In some implementations, the coherence-based measurements provide range measurements based on the interference between a projected wave and the same wave reflected/scattered back. Such measurements may be used to creates 3D volumetric scans of the eye, based on measuring through tissues and providing information about the eye structures including their thickness. For example, thickness information may be provided with less than 10 μm accuracy. In some implementations, the coherence-based measurements include scanning performed in multiple directions and/or using multiple techniques. For example, the scanning may include an A-scan, along a Z/depth axis, and/or a B-scan, along an X axis and a Y axis. A B-scan may be performed using a micromirror (MEMS mirror). In some implementations, a scan, such as an A-scan, is performed by moving a reference mirror.

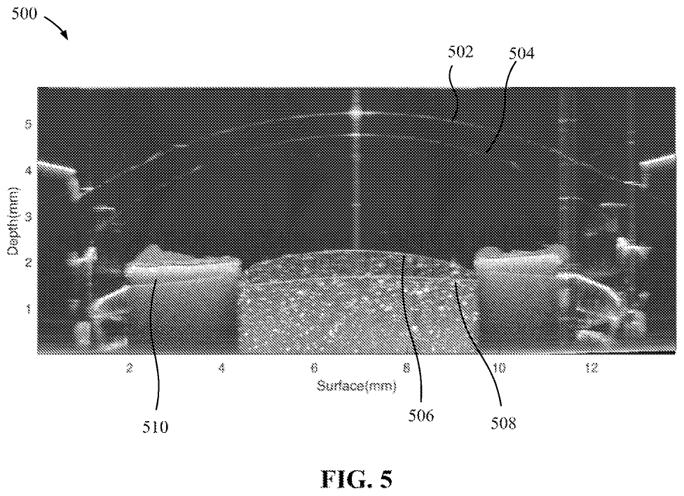

FIG. 5 illustrates an image 500 depicting data from exemplary coherence-based measurements of an eye 300 produced in accordance with some implementations. In this example, the image 500 includes a depiction 502 of a front surface of the cornea 305, a depiction 504 of the back surface of the cornea 305, a depiction 506 of the front surface of the lens 315, a depiction 508 of the back surface of the lens 315, and a depiction 510 of the shape of the iris 320. Such information representing the depths and 3D volumetric shape of aspects of the eye 300.

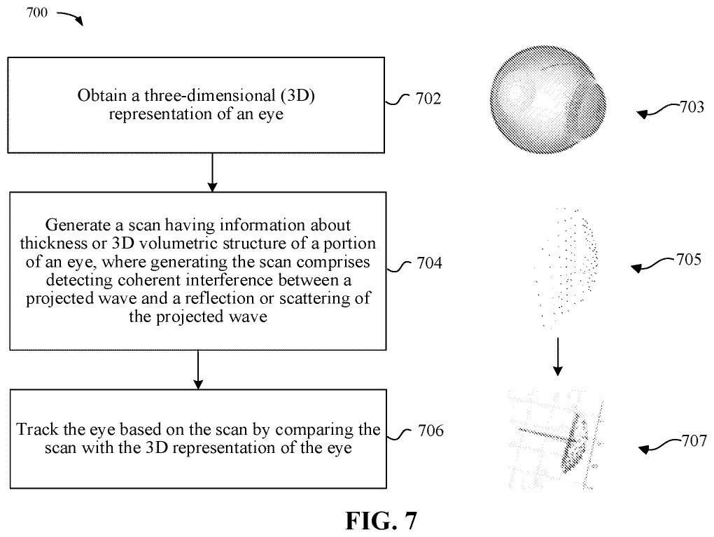

Some implementations disclosed herein track eye characteristics (e.g., eye position and/or orientation in 6 degrees of freedom, gaze direction, eye accommodative state, pupil dilation state, etc.) based on information provided by a coherence-based measurement of the eye, e.g., using optical coherence tomography (OCT). For example, this may involve creating a 3D model of a user's eye (e.g., once in a lifetime or infrequently), performing a tracking scan using coherence-based measurement of the eye, and determining the eye characteristic based on the scan and the 3D model. An example of such a technique is discussed below with reference to FIG. 7. In some implementations, the current position and/or orientation of the eye is determined by aligning (e.g., registering) the tracking scan data with the 3D model. In some implementations, the state of a portion of the eye (e.g., eye muscles such as the ciliary muscles or other portions such as the pupil opening) are assessed by comparing the tracking scan with the 3D model. An example of detecting accommodation state of the eye is discussed next.

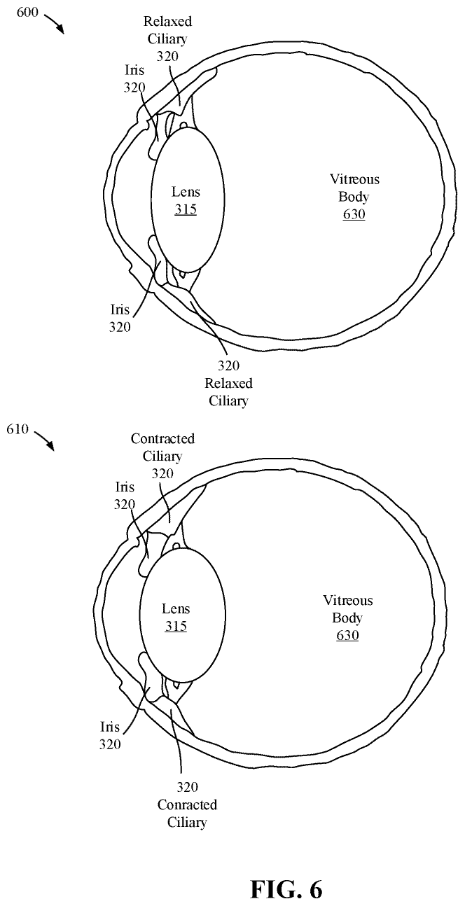

FIG. 6 illustrates ciliary differences corresponding to different accommodative states that may be detected. In this example, in state 600, the ciliary muscles 320 are relaxed and the lens 315 has a first 3D shape. In contrast, in state 610, the ciliary muscles 320 are contracted and the lens 315 has a second, different 3D shape. The ciliary muscles 320 generally contract and relax to change the shape of the lens 315 to control the user's accommodation (e.g., focus). It should be noted that the amount of lens change may vary from individual to individual and, in some cases, a user's attempt to focus using the ciliary muscles may not result in the desired change in lens 315. Some users wear corrective lenses (e.g., reading glasses, etc.) to address presbyopic conditions in which the user's eyes do not adequately accommodate/focus.

In some implementations, accommodative state of the eye 300 and/or a user's control of ciliary muscles 320 to attempt to change accommodative state is tracked by scanning the ciliary muscles 320 and/or on the lens 315. Coherence-based measurements of the eye features including the ciliary muscles 320 and/or lens 315 may be performed to assess the depth and/or 3D volumetric characteristics of these eye features, which may be interpreted to determine a user's actual and/or intended accommodative state. In one example, the actual sampled thickness of the lens 315 is compared against a 3D model of the eye 300 to determine the accommodative state of the eye 300. In another example, a user's presbyopic condition is detected based comparing sampled 3D shapes of the ciliary muscles 320 and lens 315 with the shapes of a 3D model of the eye 300. In this example, the comparison may reveal that a user's accommodative intent (as revealed by the state of the ciliary muscles 320) does not correspond to the actual amount of accommodation (as revealed by the state of the lens 315). In such cases, the device may take various response actions, for example, by adjusting the power of a corrective lens used on the device and/or changing the content to require less accommodation by the eye 300.

The shapes of the iris 320 and/or vitreous body 630 may additionally or alternatively be used to assess the user's accommodative state.

In some implementations, a user's accommodative state is tracked repeatedly over the course of a user experience using the device. For example, this may involve scanning the eye and assessing accommodative state every 1 millisecond, 10 millisecond, 100 millisecond, 1000 millisecond, etc.

In some implementations, a user's accommodative state is determined based on prior assessments of the user's eye that correspond to one or more known accommodative states. For example, during a device registration process, the user may be asked to focus on content that appears at various depths (e.g., 3 feet away, 6 feet away, 10 feet away, etc.) and scans of the user's eye while the user is focusing on content at the various distances may be obtained to generate models of the user's eye 300 corresponding to the different accommodative states. These models can then be used during later scans to assess the user's current accommodative state, e.g., by comparing against the models and/or extrapolating accommodative states between the respective model states.

FIG. 7 is a flowchart representation of a method 700 of method of tracking eye characteristics using coherence-based measurement. In some implementations, the method 700 is performed by a device (e.g., 120 of FIG. 1 or device 200 of FIG. 2), such as a head-mounted device, mobile device, desktop, laptop, or server device. In some implementations, the method 700 is performed by processing logic, including hardware, firmware, software, or a combination thereof. In some implementations, the method 700 is performed by a processor executing code stored in a non-transitory computer-readable medium (e.g., a memory).

At block 702, the method 700 obtains a 3D representation of an eye (e.g., a model 703 of the user's eye). The 3D representation may be an eye model generated based on population mean eye parameters. The 3D representation may have been generated based on a prior scan of the eye, which may be denser than subsequent scans used for eye tracking. A 3D representation may be a dense scan obtained by slow scanning to achieve high accuracy using A-scan and/or B-scan techniques. In some implementations, the 3D representation is a point cloud. In some implementations, the 3D representation is a 3D mesh, such as a mesh of triangular shapes corresponding to external and internal eye surfaces and shapes.

At block 704, the method 700 generates a scan 705 including information about the thickness or 3D volumetric structure of a portion of an eye. The scan 705 involves detecting coherent interference between a projected wave and a reflection or scattering of the projected wave. In some implementations, the scan 705 is generated by scanning processes that occur in multiple stages, for example, to conserve device resources. Such a multi-stage scan may involve and an initial scan (e.g., sparse B-scan) followed by a reduced search scan (e.g., A-scan).

In some implementations, a spatially sparse B-scan is performed initially. This may provide a sample of a sparse set of points of the eye (e.g., intercepts with cornea front and back surfaces, iris surfaces, pupil contour points, crystallin lens surfaces, ciliary muscles, points on the retina or choroid, etc.). The sparse scan may provide a grid, e.g., of 8×8 or 16×16 rays/directions. The sparse grid orientation and density may be smartly and dynamically controlled based on prior knowledge of past eye locations. A search and scan may be focused in the neighborhood of past cornea or iris locations, to obtain robust information used to determine the eye characteristics such as the eye position and/or orientation. Some eye portions may provide richer information about the position/orientation of the eye. For example, eye portions that correspond to abrupt changes in curvature, e.g., at intersection of iris plane and eye ball may provide information. Focusing a scan in those areas may produce more useful information in terms of determining the eye position/orientation or other characteristics. In another example, an iris plane may be used to generate a representative slice of the eye, which provides position/orientation and/or other eye characteristic information. Alternatively, a scan of blood vessels on the retina may be used to determine the eye position/orientation and/or other characteristics.

Given a previous scan, a subsequent reduced-scope scan may be performed. This may involve limiting or prioritizing the A-scan at expected eye surface edges, e.g., based on prior knowledge derived from recent measure eye positions/orientations. In some implementations, knowledge of eye position/orientation is used to optimize subsequent scanning, e.g., reducing the depth axis and/or density of a volumetric scan.

In some implementations, the scan identifies a sparse set of points corresponding to thickness or 3D volumetric structure of the eye portions (e.g., intercepts with cornea front/back surfaces, iris surfaces, pupil contour points, crystallin lens surfaces, ciliary muscles, points on retina or choroid, or retinal blood vessels).

In some implementations, the coherence-based measurement involves optical coherence tomography (OCT). The coherence measurement may include projecting light using a plurality of wavelengths, where a first portion of the light is directed to the eye and a second portion of the light is split off from the first portion and determining sub-surface information based on interference between: a reflection or scattering of the first portion of the light from a sub-surface structure of the eye and the second portion of the light. Such sub-surface information may include depth information, a cross section of the eye, and/or a volumetric representation of the eye. Generating the scan may involve performing a first scan to sample a set of points of the eye (e.g., a B-scan) and performing a second scan based on the first scan (e.g., an A-scan), where the first scan and second scan have different scan types.

In some implementations, the scan produces a sparse set of points based on a grid. An orientation or density of the grid may be based on prior eye location information, e.g., a prior scan that determines an approximate location and/or orientation of the eye at a prior point it time, e.g., recent enough that the eye may still be expected to be in a nearby area.

In various implementations, the scan may provide information (e.g., a set of points) corresponding to any feature of the eye including, but not limited to, a cornea, a crystallin lens, an iris, a retina, a ciliary muscle, and a choroid.

At block 706, the method 700 tracks the eye based on the scan by comparing the scan with the 3D representation of the eye. Comparison 707 illustrates points scan 705 being overlaid on points of model 703 to determine an alignment. The method 700 may track the 3D position and orientation of the eye (e.g., eye pose), gaze direction, accommodative state, pupil dilation state, etc. In some implementations, this involves performing 3D registration between sampled sparse point cloud (e.g., of block 704) with a user eye model (e.g., of block 702), e.g., via SIFT (Scale Invariant Feature Transform), RANSAC, ICP, or a machine learning model that uses, for example, a deep neural network. In some implementation, the comparison involves point cloud points. The comparison may involve determining registration scores for multiple potential alignments (e.g., registration scores) where the registration scores provide a measure of how well sampled points fit relative to the model in a given alignment and identifying an alignment with a minimum registration score.

The method 700 may continue to track the 3D position and orientation or other characteristics of the eye over time based on coherence-based measurements. In some implementations, the 3D representation is updated over time based on more recently obtained scan data, e.g., integrating deviations of sparse measurements relative the 3D representation over time.

In some implementations, the tracking of the eye is additionally based on other tracking techniques including, but not limited to, glint-reflection from front eye surfaces and/or retinal imaging techniques. The combination of multiple techniques and/or fusing of data from different sensor types may provide for robust and efficient eye tracking.

In some implementations, tracking gaze is additionally based on vergence—the concurrent movement of both of a user's eyes in opposite directions to obtain or maintain single binocular vision.

In some implementation, the position and orientation of the eye are used to determine a gaze direction. Gaze direction can be used for numerous purposes. In one example, the gaze direction that is determined or updated is used to identify an item displayed on a display, e.g., to identify what button, image, text, or other user interface item a user is looking at. In another example, the gaze characteristic that is determined or updated is used to display a movement of a graphical indicator (e.g., a cursor or other user-controlled icon) on a display. In another example, the gaze characteristic that is determined or updated is used to select an item (e.g., via a cursor selection command) displayed on a display. For example, a particular gaze movement pattern can be recognized and interpreted as a particular command.

In some implementations, the gaze tracking is performed on two eyes of a same individual concurrently. In implementations in which images of both eyes are captured or derived, the system may determine or produce output useful in determining a convergence point of gaze directions from the two eyes. The system could additionally or alternatively be configured to account for extraordinary circumstances such as optical axes that do not align.

In some implementations, post-processing of gaze direction is employed. Noise in the tracked gaze direction can be reduced using filtering and prediction methods, for example, using a Kalman filter. These methods can also be used for interpolation/extrapolation of the gaze direction over time. For example, the methods can be used if the state of the gaze direction is required at a timestamp different from the recorded states.

In some implementations, the scans of the user's eye(s) provide movement information such as measures of velocity. Such movement information may be based on frame-by-frame differences in eye portions. Velocity and other movement information may be used to further reduce the amount of scanning that is required to track the eye characteristics by providing richer information from which the eye characteristic may be tracked. Velocity and other movement information may additionally be useful in predicting saccades. Foveated rendering changes may be timed to occur during saccades. Velocity and other movement information may additionally be useful in predicting where eye movements will stop, e.g., where a user's gaze land, which may be used in reducing system latency and improving system responsiveness.

The eye tracking techniques disclosed herein may provide various advantages, e.g., over existing techniques that asses the front surfaces of the eye using glint detection or fundus using retina tracking. Some of the techniques disclosed herein may be less sensitive to interference from ambient light (e.g., sunlight) due to the coherence-based nature of the measurements. Some of the techniques disclosed herein may be less sensitive to eye lid and lashes occlusion. Some of the techniques disclosed herein may require no or less registration time in comparison to prior techniques which may require a user to fixate on a set of locations and/or follow a moving object with their eyes. In contrast, coherence-based techniques may obtain registration information (e.g., a 3D representation of the eye) without requiring eye movement or user actions. Additionally, some of the techniques disclosed herein may be used with a single scanner/sensor module, thus reducing the size requirements of the components and the overall device and/or facilitating easier integration into devices such as HMDs, including AR glasses type devices, for which a compact size may be desirable.

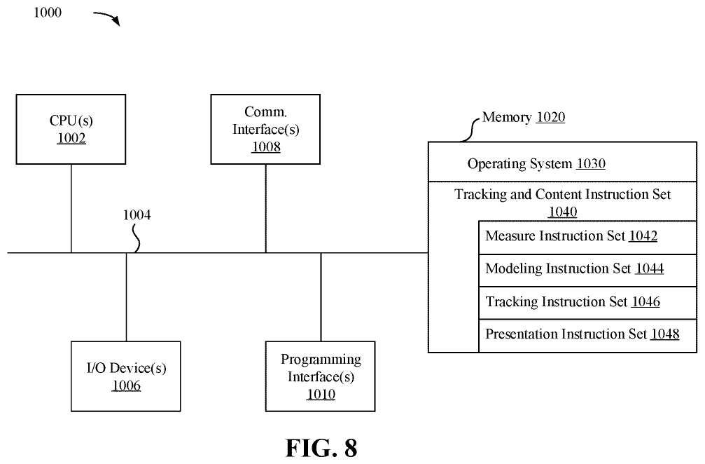

FIG. 8 is a block diagram of an example of the device 1000 in accordance with some implementations. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 1000 includes one or more processing units 1002 (e.g., microprocessors, ASICs, FPGAs, GPUs, CPUs, processing cores, and/or the like), one or more input/output (I/O) devices and sensors 1006, one or more communication interfaces 1008 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 1010, one or more displays 1012, one or more interior and/or exterior facing image sensor systems 1014, a memory 1020, and one or more communication buses 1004 for interconnecting these and various other components.

In some implementations, the one or more communication buses 1004 include circuitry that interconnects and controls communications between system components. In some implementations, the one or more I/O devices and sensors 1006 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 1012 are configured to present the experience to the user. In some implementations, the one or more displays 1012 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 1012 correspond to diffractive, reflective, polarized, holographic, etc. waveguide displays. For example, the device 1000 includes a single display. In another example, the device 1000 includes a display for each eye of the user. In some implementations, the one or more displays 1012 are capable of presenting SR content.

In some implementations, the one or more image sensor systems 1014 are configured to obtain image data that corresponds to at least a portion of the face of the user that includes the eyes of the user. For example, the one or more image sensor systems 1014 include one or more RGB cameras (e.g., with a complimentary metal-oxide-semiconductor (CMOS) image sensor or a charge-coupled device (CCD) image sensor), monochrome cameras, IR cameras, event-based cameras, and/or the like. In various implementations, the one or more image sensor systems 1014 further include illumination sources that emit light upon the portion of the face of the user, such as a flash or a glint source.

The memory 1020 includes high-speed random-access memory, such as DRAM, SRAM, DDR RAM, or other random-access solid-state memory devices. In some implementations, the memory 1020 includes non-volatile memory, such as one or more magnetic disk storage devices, optical disk storage devices, flash memory devices, or other non-volatile solid-state storage devices. The memory 1020 optionally includes one or more storage devices remotely located from the one or more processing units 1002. The memory 1020 comprises a non-transitory computer readable storage medium. In some implementations, the memory 1020 or the non-transitory computer readable storage medium of the memory 1020 stores the following programs, modules and data structures, or a subset thereof including an optional operating system 1030 and a tracking and content instruction set 1040.

The operating system 1030 includes procedures for handling various basic system services and for performing hardware dependent tasks. In some implementations, the tracking and content instruction set 1040 is configured to present content to the user via the one or more displays 1012 based on tracking the user. To that end, in various implementations, the tracking and content instruction set 1040 includes measure instruction set 1042, modeling instruction set 1044, tracking instruction set 1046, and presentation instruction set 1048. The measure instruction set 1042 is configured to perform coherence-based measurements of one or more eyes as described herein. The modeling instruction set 1044 is configured to generate 3D representations of the one or more eyes, for example, using eye measurements and/or standard eye model information as disclosed herein. The tracking instruction set is configured to track one or more eye characteristics based on eye measurement data and/or 3D representations as described herein. The presentation instruction set 1048 is configured to present views of physical environments and/or virtual environments (e.g., extended reality XR content) and/or to provide content or otherwise control a user experience based on the tracking of the user's eye characteristics.

Although these elements are shown as residing on a single device, it should be understood that in other implementations, any combination of the elements may be located in separate computing devices. Moreover, FIG. 8 is intended more as functional description of the various features which are present in a particular implementation as opposed to a structural schematic of the implementations described herein. As recognized by those of ordinary skill in the art, items shown separately could be combined and some items could be separated. For example, some functional modules shown separately in FIG. 8 could be implemented in a single module and the various functions of single functional blocks could be implemented by one or more functional blocks in various implementations. The actual number of modules and the division of particular functions and how features are allocated among them will vary from one implementation to another and, in some implementations, depends in part on the particular combination of hardware, software, and/or firmware chosen for a particular implementation.

Numerous specific details are set forth herein to provide a thorough understanding of the claimed subject matter. However, those skilled in the art will understand that the claimed subject matter may be practiced without these specific details. In other instances, methods apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general-purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

1. A method comprising:
at an electronic device having a processor:
obtaining a three-dimensional (3D) representation of an eye;
generating a scan comprising information about thickness or 3D volumetric structure of a portion of an eye, wherein generating the scan comprises detecting coherent interference between a split off wave of a projected wave and a reflection or scattering of the projected wave; and
tracking the eye based on the scan by comparing the scan with the 3D representation of the eye.

2. The method of claim 1, wherein tracking the eye comprises determining a 3D position and orientation of the eye.

3. The method of claim 2 further comprising continuing to track the 3D position and orientation of the eye over time based on coherence-based measurements.

4. The method of claim 1, wherein tracking the eye comprises determining a state of accommodation of the eye.

5. The method of claim 1, wherein tracking the eye comprises determining a dilation state of a pupil of the eye.

6. The method of claim 1, wherein the coherence-based measurement comprises optical coherence tomography (OCT).

7. The method of claim 1, wherein the coherence measurement comprises:
projecting light using a plurality of wavelengths, wherein a first portion of the light is directed to the eye and a second portion of the light is split off from the first portion; and
determining sub-surface information based on interference between:
a reflection or scattering of the first portion of the light from a sub-surface structure of the eye; and
the second portion of the light.

8. The method of claim 6, wherein the sub-surface information comprises depth information, a cross section of the eye, or a volumetric representation of the eye.

9. The method of claim 1, wherein generating the scan comprises:
performing a first scan to sample a set of points of the eye;
performing a second scan based on the first scan, wherein the first scan and second scan have different scan types.

10. The method claim 1, wherein the scan produces a sparse set of points based on a grid, wherein an orientation or density of the grid is based on prior eye location information.

11. The method of claim 1, wherein the scan identifies a set of points corresponding to a portion of the eye comprising:
a cornea;
a crystallin lens;
an iris;
a retina;
a ciliary muscle; or
a choroid.

12. The method of claim 1, wherein the 3D representation was generated based on a prior scan of the eye.

13. The method of claim 12, wherein the prior scan is denser than the scan.

14. The method of claim 1, wherein the 3D representation is an eye model generated based on population mean eye parameters.

15. The method of claim 1, wherein the device is a head-mounted device (HMD).

16. The method of claim 1 further comprising providing content based on the eye tracking.

17. The method of claim 1, wherein the eye tracking is further based on a glint-reflection or retinal imaging technique.

18. An electronic device comprising:
a wave source configured to project waves using a plurality of wavelengths, wherein the wave source is configured to direct a first portion of the waves toward the eye and split off a second portion of the waves;
a sensor configured to capture the waves reflected or scattered by the eye;
a non-transitory computer-readable storage medium; and
one or more processors coupled to the non-transitory computer-readable storage medium, wherein the non-transitory computer-readable storage medium comprises program instructions that, when executed on the one or more processors, cause the device to perform operations comprising:
obtaining a three-dimensional (3D) representation of an eye;
generating a scan comprising information about thickness or 3D volumetric structure of a portion of an eye, wherein generating the scan comprises detecting coherent interference between a split off wave of a projected wave and a reflection or scattering of the projected wave; and
tracking the eye based on the scan by comparing the scan with a 3D representation of the eye.

19. The electronic device of claim 18, wherein tracking the eye comprises determining a 3D position and orientation of the eye.

20-34. (canceled)

35. A non-transitory computer-readable storage medium storing instructions executable on an electronic device to perform operations comprising:
obtaining a three-dimensional (3D) representation of an eye;
generating a scan comprising information about thickness or 3D volumetric structure of a portion of an eye, wherein generating the scan comprises detecting coherent interference between a split off wave of a projected wave and a reflection or scattering of the projected wave; and
tracking the eye based on the scan by comparing the scan with a 3D representation of the eye.

36-51. (canceled)
